(19) United States
(12) Patent Application Publication
(10) Pub. No.: US 2007/0049752 A1
(43) Pub. Date: Mar. 1, 2007

(54) Preparation of 2,6-dioxa-7-aza-bicyclo[2.2.2] octanes

(76) Inventor: Neville Everton Drysdale, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/506,021

(22) Filed: Aug. 17, 2006

Related U.S. Application Data
(60) Provisional application No. 60/710,360, filed on Aug. 23, 2005.
(60) Provisional application No. 60/710,362, filed on Aug. 23, 2005.
(60) Provisional application No. 60/710,358, filed on Aug. 23, 2005.

(51) Int. Cl. C07D 498/02

(52) U.S. Cl. 544/105

(57) ABSTRACT

The preparation of 2,6-dioxa-7-aza-bicyclo[2.2.2]octane is disclosed herein. These materials find use in a variety of applications, including coatings, which generally have lower volatile organic compounds (VOCs).

### CROSS-REFERENCE APPLICATIONS

This Application claims the priority benefit of U.S. provisional patent application 60/710360, filed Aug. 23, 2005; and U.S. provisional patent application 60/710362, filed Aug. 23, 2005; and U.S. provisional patent application 60/710358, filed Aug. 23, 2005.

### FIELD OF THE INVENTION

The preparation of 2,6-dioxa-7-aza-bicyclo[2.2.2]octane, is disclosed herein. These materials find use in a variety of applications, including coatings, which have lower levels of volatile organic compounds (VOCs).

### BACKGROUND OF THE INVENTION

There is a need for film-forming materials that can be part of low VOC (volatile organic compounds) cross-linked systems for coating and other applications. Such materials generally react and cross-link through non-volatile ring-opening additions with poly(isocyanates) to form these coatings, which exhibit desirable final product properties. Generally, by “low VOC” is meant less than about 2.1 pounds of applicable organic solvent(s) is contained in a gallon of paint, as determined under the procedure provided in ASTM D3960.

One class of these materials is 2,6-dioxa-7-aza-bicyclo[2.2.2]octanes. S. Kanoh, et al., J. Org. Chem, 2000, 65, 2253-56 describes a method of preparation of substituted 2,6-dioxa-7-aza-bicyclo[2.2.2]octanes. However, the disclosed synthetic routes comprise at least five steps, starting from 3-alkyl-3-(hydroxymethyl)-oxetane, and thus the yield of desired end-product is quite low.

A simplified, more efficient, process to produce 2,6-dioxa-7-aza-bicyclo[2.2.2]octanes is needed to provide sufficient desired components for coatings.

### SUMMARY OF THE INVENTION

Disclosed herein is a process for forming a 2,6-dioxa-7-aza-bicyclo[2.2.2]octane, said process comprising: combining a 2-alkyl-2-alkylaminomethyl-propane-1,3-diol with an orthoester, optionally in the presence of a solvent, and optionally in the presence of an acid, to form a mixture comprising an alcohol; and distilling said alcohol in an inert atmosphere to produce a 2,6-dioxa-7-aza-bicyclo[2.2.2]octane.

Also disclosed herein is the above process further comprising, after step b), neutralizing said mixture with an amine and isolating said 2,6-dioxa-7-aza-bicyclo[2.2.2]octane.

A further disclosure is a process for production of 2,6-dioxa-7-aza-bicyclo[2.2.2]octane, said process comprising:
a) reacting a 2-alkyl-2-alkylaminomethyl-propane-1,3-diol with a nitrile in the presence of a metal salt, optionally in the presence of a solvent, to form a mixture comprising ammonia; and
b) removing said ammonia to produce a 2,6-dioxa-7-aza-bicyclo[2.2.2]octane.

The present disclosure further describes a composition, comprising: one or more 2,6-dioxa-7-aza-bicyclo[2.2.2]octanes of the structure 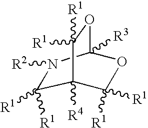
wherein each R¹, R², R³ and R⁴ can independently be C1-C20 alkyl, C6-C20 aromatic, C6-C20 alkylaromatic or C6-C20 aromaticalkyl, wherein each of R¹, R², R³ and R⁴ may be substituted with hydroxyl, amino, epoxy, or carboxy groups; and

a crosslinking moiety.

It further relates to the process of making a coating comprising the 2,6-dioxa-7-aza-bicyclo[2.2.2]octanes and a crosslinking moiety, and coating systems comprising such materials.

### DETAILED DESCRIPTION OF THE INVENTION

Described herein is the production of 2,6-dioxa-7-aza-bicyclo[2.2.2]octanes in an efficient manner. Previously, these compounds have been synthesized by reaction schemes involving five or more steps, and generally are low-yield procedures.

New, efficient synthetic routes for the production of 2,6-dioxa-7-aza-bicyclo[2.2.2]octanes, shown generally in structure 1 below, have been discovered. 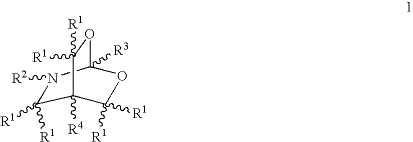
As shown in structure 1, each R¹, R², R³ and R⁴ can be independently C1-C20 alkyl, C6-C20 aromatic, C6-C20 alkylaromatic or C6-C20 aromaticalkyl, wherein each of R¹, R², R³ and R⁴ may be substituted with hydroxyl, amino, epoxy, or carboxy groups.

One novel preparation first involves the reaction of 3-alkyl-3-(hydroxymethyl)-oxetane with an alkylamine to give a 2-alkyl-2-alkylaminomethyl-propane-1,3-diol, as shown in Reaction Scheme 1, and described in A Jacobi, et al., U. Chem ber, Recueil 1997, 130,1279-1294.

Generally, the starting material 3-alkyl-3-(hydroxymethyl)-oxetane is produced according to the method described by J. V. Crivello and H. Sasake, J. Macromolecular Sci., Pure and Applied Chemistry, 1993, A30(2-3), 189-206, or purchased from Perstorp, Toledo, Ohio. It is generally preferred that the 3-alkyl-3-(hydroxymethyl)-oxetane have an alkyl carbon chain length of 1 to 20. It is also preferred that the carbon chain length of said 2-alkyl-2-alkylaminomethyl-propane-1,3-diol has a carbon chain length of 1 to 20. 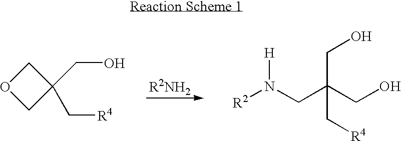
Subsequently, the 2-alkyl-2-alkylaminomethyl-propane-1,3-diol is then reacted with trialkyl orthoesters as depicted in Reaction Scheme 2 below to give the desired 2,6-dioxa-7-aza-bicyclo[2.2.2]octane. 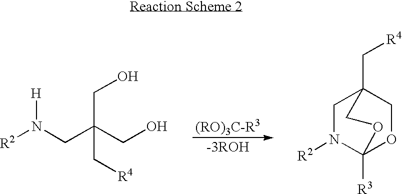
Therefore, the desired end-products are made from the commercially available oxetane in two steps, with relatively high yields.

The resulting substituted diol is then combined with an orthoester, optionally in the presence of a solvent, heated with stirring to remove the alcohol formed. The reaction is generally performed in the presence of a strong acid, such as toluenesulfonic acid. After the theoretical amount of alcohol is distilled off the reaction is cooled to room temperature and then a small amount of trialkylamine is added to neutralize the acid. The product can then be isolated by any method known to those skilled in the art. Examples of isolation methods include distillation and crystallization. As shown in the Examples herein, fractional vacuum distillations afford the desired material as a liquid, generally water-clear.

A second novel preparation of 2,6-dioxa-7-aza-bicyclo[2.2.2]octanes first involves the reaction of 3-alkyl-3-(hydroxymethyl)-oxetane with an alkylamine to give a 2-alkyl-2-alkylaminomethyl-propane-1,3-diol, as shown in Reaction Scheme 1 above, followed by a subsequent reaction of the 2-alkyl-2-alkylaminomethyl-propane-1,3-diol with a nitrile in the presence of a metal salt as depicted in Reaction Scheme 4 below to give the desired 2,6-dioxa-7-aza-bicyclo[2.2.2]octane. 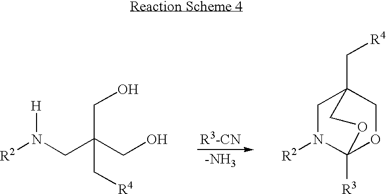
Therefore, the desired end-products are made from the commercially available oxetane in two steps, with relatively high yields.

The resulting substituted diol is then combined with a nitrile, in the presence of a metal salt, and optionally in the presence of a solvent, heated with stirring to remove the ammonia formed. The product can then be isolated by any method known to those skilled in the art. Examples of isolation methods include distillation and crystallization. As shown in the Examples below, fractional vacuum distillations afford the desired material as a liquid.

Any 2-alkyl-2-alkylaminomethyl-1,3-propanediols can be used in the present disclosure. Non-limiting examples of such diols include 2-butyl-2-(methylaminomethyl)propanediol, and 2-hexyl-2-(methylaminomethyl)propanediol.

Many orthoesters can be used as described herein. Non-limiting examples of such orthoesters include trimethyl orthoacetate, triethyl orthoacetate and ortho-n-valeric acid trimethyl ester.

Any nitrites can be used as described herein. Alkyl nitriles with a carbon chain length of 1 to 20 are preferred.

Metal salts are used in a catalytic fashion as described herein. Generally, the catalyst is a salt of a principal metal component, wherein the principal metal component is selected from the group consisting of scandium, titanium, vanadium, chromium, manganese, iron, cobalt, nickel, copper, zinc, and cadmium. Preferred metal salts include zinc acetate dihydrate and cadmium acetate dihydrate.

Solvents can optionally be used as described herein. Such solvents are generally non reactive. Non-limiting examples of such solvents include toluene and xylene.

Amines that can be used as described herein include trialkylamines and pyridine. Triethylamine is generally preferred.

The 2,6-dioxa-7-aza-bicyclo[2.2.2]octanes thus formed by the novel process as described herein find use in formulating low volatile organic compound (VOC) coatings. These coatings are useful in the automotive and architectural markets.

The present disclosure further relates to the use of 2,6-dioxa-7-aza-bicyclo[2.2.2]octanes of the general structure 1 with crosslinkers, e.g., multifunctional isocyanates, to provide crosslinked coatings having low volatile organic compound (VOC) levels. 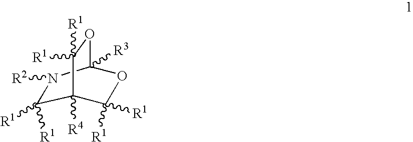
As shown in structure 1, each R¹, R², R³ and R⁴ can be independently C1-C20 alkyl, C6-C20 aromatic, C6-C20 alkylaromatic or C6-C20 aromaticalkyl, wherein each of R¹, R², R³ and R⁴ may be substituted with hydroxyl, amino, epoxy, or carboxy groups.

The crosslinking (curing) of polymers is an important commercial activity, useful, for example, in elastomers, in coatings, and in thermoset materials such as are used for electronics. Controlling when and under what conditions crosslinking takes place is usually critical since once a polymer is crosslinked it is usually not “workable,” that is it may not be reshaped. In some applications, such as coatings and electronic applications it may be desirable or even mandatory that no lower molecular weight compounds be volatilized during or after the crosslinking of the polymers, so as not to contaminate sensitive equipment such as electronics, and/or to pollute the environment, as in the case of coatings.

Numerous ways have been found to avoid the production of volatile compounds during curing. For example, the ring-opening reaction of epoxy groups with other groups such as hydroxyl groups may accomplish this result, but it is sometimes difficult to control after the ingredients are mixed. Furthermore, higher temperatures may be required for this operation. To avoid these types of problems, especially in coatings which often must be cured under conditions close to ambient conditions and which often must be stable for long periods before curing, other solutions have been found, such as the use of spiroorthoesters, see for example PCT Application WO9731073. However new and/or improved methods of crosslinking polymers are needed.

For coatings, basecoat-clearcoat systems have found wide acceptance in the past decade as automotive finishes. Continuing effort has been directed to such coating systems to improve the overall appearance, the clarity of the topcoat, and the resistance to deterioration. Further effort has been directed to the development of coating compositions having low volatile organic content (VOC). A continuing need exists for coating formulations which provide outstanding performance characteristics after application.

In repairing damage, such as dents to auto bodies, the original coating in and around the damaged area is typically sanded or ground out by mechanical means. Sometimes the original coating is stripped off from a portion or off the entire auto body to expose the bare metal underneath. After repairing the damage, the repaired surface is coated, preferably with low VOC coating compositions, typically in portable or permanent low cost painting enclosures, vented to atmosphere to remove the organic solvents from the freshly applied paint coatings in an environmentally safe manner. Typically, the drying and curing of the freshly applied paint takes place within these enclosures. Furthermore, the foregoing drying and curing steps take place within the enclosure to also prevent the wet paint from collecting dirt or other contaminants in the air.

As these paint enclosures take up significant floor space of typical small auto body paint repair shops, these shops prefer to dry and cure these paints as fast as possible. More expensive enclosures are frequently provided with heat sources, such as conventional heat lamps located inside the enclosure to cure the freshly applied paint at accelerated rates. Therefore, to provide more cost effective utilization of shop floor space and to minimize fire hazards resulting from wet coatings from solvent based coating compositions, there exists a continuing need for low VOC fast curing coating formulations which cure under ambient conditions while still providing outstanding performance characteristics.

By polymers herein are meant those entities with number average molecular weight from about 100 to about 100,000. Preferably, the number average molecular weight of the polymers is from about 100 to about 10000.

By oligomers herein is meant those polymers which have a number average molecular weight less than about 3000.

In the crosslinkable compositions herein, 2,6-dioxa-7-aza-bicyclo[2.2.2]octanes groups are present and the crosslinking reaction can be initiated when water comes in contact with these groups to hydrolyze them. By water is meant water in the pure form, moisture, moist air, moist gas or mixture of gases, or any other aqueous or non-aqueous media in which water may be present in a homogeneous or a heterogeneous mixture. Such media may be in the liquid form or the gaseous form.

When the 2,6-dioxa-7-aza-bicyclo[2.2.2]octane is hydrolyzed, amino hydroxy ester is formed which then converts to the amide diol as illustrated below. The amino hydroxy ester and the amide diol exist simultaneously in the reaction and the rate of conversion of the amino hydroxy ester to amide diol can be controlled by time, temperature, pH, and the urethane forming catalyst present in the reaction mixture. An advantage of the amide diol is that it demonstrates minimal yellowing in the finished product, before reacting with crosslinking agent. A rapid reaction with the cross-linking agent avoids the yellowing of the amine functionality in the product. Both of these hydrolyzed products are cross-linking agents because of the presence of their dual reactive side. In the case of the amino hydroxy ester the reactive sites are the secondary amine and the hydroxyl groups. In the case of the amide diol the reactive groups are the hydroxyls or diol:

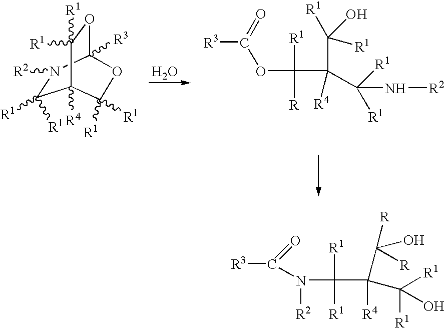

No relatively volatile low molecular weight products are produced, i.e., no harmful volatile by-products are released. Since these reactions may optionally be acid catalyzed some of the ring opening of the 2,6-dioxa-7-aza-bicyclo[2.2.2]octane may lead to cationic polymerization rather than simple ring opening. Herein preferably the major molar portion of the 2,6-dioxa-7-aza-bicyclo[2.2.2]octane present may ring open and polymerize, preferably at least about 75 mole percent, and more preferably at least 90 molar percent may ring open and polymerize. The polymerization occurs generally at high temperatures.

In the compositions, and in the materials used in the processes herein, the 2,6-dioxa-7-aza-bicyclo[2.2.2]octane groups may be included by a variety of methods. In one instance, the 2,6-dioxa-7-aza-bicyclo[2.2.2]octane may be included as a “monomeric” compound, which may hydrolyze, thus providing reactive hydroxyl groups.

In another embodiment, one can form a polymer, preferably an acrylic polymer having isocyanate groups (from, for example, 2-isocyanatoethyl (meth)acrylate). This acrylic polymer can then be reacted with a 2,6-dioxa-7-aza-bicyclo[2.2.2]octane moiety, wherein one of the R or R¹ groups contains a hydroxyl, amine or carboxy group. The acrylic polymer will have at least one 2,6-dioxa-7-aza-bicyclo[2.2.2]octane, but may and preferably will have more than one. The number of 2,6-dioxa-7-aza-bicyclo[2.2.2]octane groups present in the acrylic polymer will depend on the reaction stoichiometry and the amount of isocyanate groups present in the acrylic polymer.

Alternatively, the 2,6-dioxa-7-aza-bicyclo[2.2.2]octane groups may be part of a polymer. For example a dihydroxy 2,6-dioxa-7-aza-bicyclo[2.2.2]octane (which has not yet been hydrolyzed) may be reacted with an excess of a diisocyanate such as bis(4-isocyanatophenyl)methane (MDI), toluene diisocyanate (TDI), hexamethylene diisocyanate (HMDI) or isophorone diisocyanate (IPDI) to form an isocyanate ended “prepolymer”, which upon exposure to water undergoes hydrolysis of the 2,6-dioxa-7-aza-bicyclo[2.2.2]octane forming hydroxyl groups, which react with the remaining isocyanate groups to crosslink the polymer. Since 2,6-dioxa-7-aza-bicyclo[2.2.2]octane often hydrolyze faster than isocyanate reacts with water, this is believed to be main mode of the crosslinking reaction for this type of polymer. Other diols such as ethylene glycol or 1,4-butanediol may also be copolymerized into the (pre)polymer formed. It is noted that in this type of isocyanate containing (pre)polymer, the 2,6-dioxa-7-aza-bicyclo[2.2.2]octane group is (at least before hydrolysis) part of the main chain (not on a branch) of the polymer formed.

Alternately, the 2,6-dioxa-7-aza-bicyclo[2.2.2]octane may be functionalized, for example, via reaction of (mono)hydroxy 2,6-dioxa-7-aza-bicyclo[2.2.2]octane with isocyanate to give urethane 2,6-dioxa-7-aza-bicyclo[2.2.2]octane, or with diisocyanates, for example, 1,6-hexamethylene diisocyanate, to give diurethane 2,6-dioxa-7-aza-bicyclo[2.2.2]octanes, or DESMODUR 3300 which contains multifunctional isocyanates, a triisocyanate, to give the corresponding multifunctional urethane 2,6-dioxa-7-aza-bicyclo[2.2.2]octanes. Many of these compounds are novel.

An example of the cross-linking agent, or second polymer with functional groups capable of reacting with hydroxyl or secondary amines, for the 2,6-dioxa-7-aza-bicyclo[2.2.2]octane is as follows: 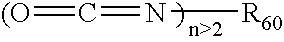
wherein R₆₀ is a hydrocarbyl structure.

Examples of suitable polyisocyanates include aromatic, aliphatic or cycloaliphatic di-, tri- or tetra-isocyanates, including polyisocyanates having isocyanurate structural units, such as, the isocyanurate of hexamethylene diisocyanate and isocyanurate of isophorone diisocyanate; the adduct of 2 molecules of a diisocyanate, such as, hexamethylene diisocyanate and a diol such as, ethylene glycol; uretidiones of hexamethylene diisocyanate; uretidiones of isophorone diisocyanate or isophorone diisocyanate; the adduct of trimethylol propane and meta-tetramethylxylylene diisocyanate.

Additional examples of suitable polyisocyanates include 1,2-propylene diisocyanate, trimethylene diisocyanate, tetramethylene diisocyanate, 2,3-butylene diisocyanate, hexamethylene diisocyanate, octamethylene diisocyanate, 2,2,4-trimethyl hexamethylene diisocyanate, 2,4,4-trimethyl hexamethylene diisocyanate, dodecamethylene diisocyanate, omega, omega -dipropyl ether diisocyanate, 1,3-cyclopentane diisocyanate, 1,2-cyclohexane diisocyanate, 1,4-cyclohexane diisocyanate, isophorone diisocyanate, 4-methyl-1,3-diisocyanatocyclohexane, trans-vinylidene diisocyanate, dicyclohexylmethane-4,4′-diisocyanate, 3,3′-dimethyl-dicyclohexylmethane4,4′-diisocyanate, a toluene diisocyanate, 1,3-bis(1-isocyanato1-methylethyl)benzene, 1,4-bis(1-isocyanato-1-methylethyl)benzene, 1,3-bis(isocyanatomethyl)benzene, xylene diisocyanate, 1,5-dimethyl-2,4-bis(isocyanatomethyl)benzene, 1,5-dimethyl-2,4-bis(2-isocyanatoethyl)benzene, 1,3,5-triethyl-2,4-bis(isocyanatomethyl)benzene, 4,4′-diisocyanatodiphenyl, 3,3′-dichloro-4,4′-diisocyanatodiphenyl, 3,3′-diphenyl-4,4′-diisocyanatodiphenyl, 3,3′-dimethoxy-4,4′-diisocyanatodiphenyl, 4,4′-diisocyanatodiphenylmethane, 3,3′-dimethyl-4,4′-diisocyanatodiphenyl methane, a diisocyanatonaphthalene, polyisocyanates having isocyanaurate structural units, the adduct of 2 molecules of a diisocyanate, such as, hexamethylene diisocyanate or isophorone diisocyanate, and a diol such as ethylene glycol, the adduct of 3 molecules of hexamethylene diisocyanate and 1 molecule of water (available under the trademark Desmodur® N from Bayer Corporation of Pittsburgh, Pa.), the adduct of 1 molecule of trimethylol propane and 3 molecules of toluene diisocyanate (available under the trademark Desmodur® L from Bayer Corporation), the adduct of 1 molecule of trimethylol propane and 3 molecules of isophorone diisocyanate, compounds such as 1,3,5-triisocyanato benzene and 2,4,6-triisocyanatotoluene, and the adduct of 1 molecule of pentaerythritol and 4 molecules of toluene diisocyanate.

In one instance a first polymer containing intact (before hydrolysis) 2,6-dioxa-7-aza-bicyclo[2.2.2]octane groups, and a crosslinking agent containing functional groups react with hydroxyl or secondary amine groups. The crosslinking agent may be a monomeric compound such as a diisocyanate such as MDI, TDI, HMDI or IPDI, or an epoxy resin, or may be a polymer containing functional groups. For example it may be (meth)acrylate copolymer containing repeat units derived from 2-isocyanatoethyl (meth)acrylate or glycidyl (meth)acrylate. It is also possible that the first polymer and the crosslinking agent are “combined” in the same polymer. For example one can copolymerize an 2,6-dioxa-7-aza-bicyclo[2.2.2]octane with 2-isocyanatoethyl (meth)acrylate or glycidyl (meth)acrylate and optionally other copolymerizable monomers. When that single polymer is exposed to moisture, the 2,6-dioxa-7-aza-bicyclo[2.2.2]octane groups will hydrolyze forming amino hydroxy groups (which convert to hydroxyl groups as shown previously), which in turn will react with the isocyanate, carboxylic acid anhydride, melamine, silane(s) or epoxide groups, whichever are present. These materials may be combined as a single polymer or may be more than one substance.

In one embodiment as described herein, a second polymer which has functional groups capable of reacting with hydroxyl or secondary amines has a number average molecular weight less than 3000. A preferred functionality for this second polymer is isocyanate.

A specific example of the cross-linking agent, or second polymer with functional groups capable of reacting with hydroxyl or secondary amines, used here is the Desmodur® 3300 compound from Bayer. The idealized structure of Desmodur® 3300 is given as follows (also, pentamer, heptamer and higher molecular weight species can be present):

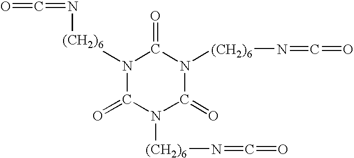

Also present in these compositions, as 2,6-dioxa-7-aza-bicyclo[2.2.2]octanes and the processes in which they are used, is a material having functional groups that react with hydroxyl or secondary amine groups. This reaction should take place under the conditions chosen for the crosslinking reaction. Ambient, heating or other conditions may be used to initiate the reaction. Preferably the reaction with hydroxyl or secondary amine groups should not produce any volatile low molecular weight compounds, except those normally found in air (CO₂, water, etc.). Typical groups which react with hydroxyl or secondary amine groups include isocyanates (including isocyanurate trimers), epoxides, carboxylic acid anhydrides (especially those which are parts of polymers), melamine, carbamates, and silane(s). Isocyanates, melamine and silane are especially preferred for coatings. Typically the ratio of equivalents of isocyanate to amine and/or hydroxyl is between 1:1 and 1.8:1.

In any of the compositions herein, the polymeric materials may range from relatively low to relatively high molecular weight. It is preferred that they be of relatively low molecular weight so as to keep the viscosity of the compositions before crosslinking low, so as to avoid or minimize the need for solvent(s).

The compositions herein may contain water. It is to be understood that as the water contacts the 2,6-dioxa-7-aza-bicyclo[2.2.2]octane groups present in the composition, the 2,6-dioxa-7-aza-bicyclo[2.2.2]octane groups will start to hydrolyze, eventually leading to crosslinking of the composition. The water may be introduced in a variety of ways. For example, especially in the case of a coating the water may introduced into the uncrosslinked or crosslinking (while the crosslinking is taking place) coating by absorption from the air. This is very convenient for making an uncrosslinked coating composition which is stable until exposed to (moist) air. Alternatively water may be mixed in a mixing head or spray mixing head (for a coating) just before crosslinking is to take place. This is particularly useful for making thicker crosslinked items such as electronic encapsulants where diffusion of moisture into a thicker section will take longer. The introduction of water can be at a point where the final shape of the polymeric crosslinked part can be formed before crosslinking takes place.

Other materials which may optionally be present in the compositions and processes include one or more solvents (and are meant to act only as solvents). These preferably do not contain groups such as hydroxyl or primary or secondary amino which can react with either the first or second functional groups and/or 2,6-dioxa-7-aza-bicyclo[2.2.2]octanes. One or more catalysts for the hydrolysis of 2,6-dioxa-7-aza-bicyclo[2.2.2]octanes may be present. These are typically Brönsted acids, but these acids should not be so strong as cause substantial cationic ring opening polymerization of the 2,6-dioxa-7-aza-bicyclo[2.2.2]octanes and/or epoxides which may be present. If substantial cationic ring opening polymerization of 2,6-dioxa-7-aza-bicyclo[2.2.2]octane groups takes place, this can often lead to premature crosslinking of the composition. The same caveats may be said for any catalysts which may be present which catalyze the reaction of hydroxyl groups or the amino hydroxy groups with the first or second functional groups. What these catalysts may be will depend on what the first or second functional group(s) present are. Such catalysts are known in the art.

Some of the suitable catalysts for polyisocyanate can include one or more tin compounds, tertiary amines or a combination thereof; and one or more aforedescribed acid catalyst. Suitable tin compounds include dibutyl tin dilaurate, dibutyl tin diacetate, stannous octoate, and dibutyl tin oxide. Dibutyl tin dilaurate is preferred. Suitable tertiary amines include triethylene diamine. One commercially available catalyst that can be used is Fastcat® 4202 dibutyl tin dilaurate sold by Elf-AtoChem North America, Inc. Philadelphia, Pa. It is acknowledged that one skilled in the art could use acetic acid or such weak acids to block the activity of the catalyst.

The present compositions, and the process for making them crosslinked, are useful as encapsulants, sealants, and coatings. The coating composition can be used as a clear coat that is applied over a pigmented base coat that may a pigmented version of the disclosed composition or another type of a pigmented base coat. The clear coating can be in solution or in dispersion form.

Typically, a clear coating is then applied over the base coating before the base coating is fully cured, a so called “wet-on-wet process”, and the base coating and clear coating are then fully cured at ambient temperatures or can be cured by heating to elevated temperatures of 40° C. to 100° C. for 15 to 45 minutes. The base coating and clear coating preferably have a dry coating thickness ranging from 25 to 75 microns and 25 to 100 microns, respectively. By “crosslinker functionality” is meant is the average number of functional groups per molecule. If the functionality of the crosslinker is too low, disruption of the basecoat flake orientation may occur. This disruption is measured by flop. The higher the value of flop the lower the amount of flake orientation disruption. Less disruption of the flake orientation is seen with a crosslinker which has >3.1 average functionality and a viscosity at 100% solids at 23° C. of >700 mPas, preferably >900 mPas, and most preferably >1000 mPas. These values are measured with a color measurement device and compared to a commercial standard.

The novel coating composition may be used as a base coat or as a pigmented monocoat topcoat. Both of these compositions require the presence of pigments. Typically, a pigment-to-binder ratio of 0.1/100 to 200/100 is used depending on the color and type of pigment used. The pigments are formulated into mill bases by conventional procedures, such as, grinding, sand milling, and high speed mixing. Generally, the mill base comprises pigment and a dispersant in an aqueous medium. The mill base is added in an appropriate amount to the coating composition with mixing to form a pigmented coating composition.

Any of the conventionally-used organic and inorganic pigments, such as, white pigments, like, titanium dioxide, color pigments, metallic flakes, such as, aluminum flake, special effects pigments, such as, coated mica flakes, coated aluminum flakes and the like and extender pigments can be used. It may be desirable to add flow control additives.

The novel coating composition may be used as a primer in which case typical pigments used in primers would be added, such as, carbon black, barytes, silica, iron oxide and other pigments that are commonly used in primers in a pigment-to-binder ratio of 150/100 to 300/100.

The coating composition can be applied by conventional techniques, such as, spraying, electrostatic spraying, dipping, brushing, and flow coating.

The coating composition is particularly useful for the repair and refinish of automobile bodies and truck bodies and parts as a clear coat, pigmented base coat, or as a primer. The novel composition has uses for coating any and all items manufactured and painted by automobile sub-suppliers, frame rails, commercial trucks and truck bodies, including but not limited to beverage bodies, utility bodies, ready mix concrete delivery vehicle bodies, waste hauling vehicle bodies, and fire and emergency vehicle bodies, as well as any potential attachments or components to such truck bodies, buses, farm and construction equipment, truck caps and covers, commercial trailers, consumer trailers, recreational vehicles, including but not limited to, motor homes, campers, conversion vans, vans, large commercial aircraft and small pleasure aircraft, pleasure vehicles, such as, snow mobiles, all terrain vehicles, personal watercraft, motorcycles, and boats. The novel composition also can be used as a coating for industrial and commercial new construction and maintenance thereof; cement and wood floors; walls of commercial and residential structures, such as, office buildings and homes; amusement park equipment; concrete surfaces, such as parking lots and drive ways; asphalt and concrete road surface, wood substrates, marine surfaces; outdoor structures, such as bridges, towers; coil coating; railroad cars; printed circuit boards; machinery; OEM tools; signs; fiberglass structures; sporting goods; and sporting equipment.

This makes these coatings particularly useful for repainting of transportation vehicles in the field. An advantage of the present materials and processes in encapsulants and sealants is that when 2,6-dioxa-7-aza-bicyclo[2.2.2]octanes are used in crosslinking reactions the resulting product does not shrink, or shrink as much as usual in a typical crosslinking reaction. This means any volume to be filled by the crosslinked material will be more reliably filled with a reduced possibility of voids being present due to shrinkage during crosslinking.

For whatever uses they are put to, the compositions, and the materials used in the processes described herein may contain other materials which are conventionally used in such uses. For example, for use as encapsulants and sealants the composition may contain fillers, pigments, and/or antioxidants.

For coatings there may be a myriad of other ingredients present, some of which are described below. In particular there may be other polymers (especially of low molecular weight, “functionalized oligomers”) which are either inert or have functional group(s) other than those that may act as the materials comprising 2,6-dioxa-7-aza-bicyclo[2.2.2]octanes and also react with other reactive materials in the coating composition.

Representative of the functionalized oligomers that can be employed as components or potential cross-linking agents of the coatings are the following:

Acid Oligomers: The reaction product of multifunctional alcohols such as pentaerythritol, hexanediol, trimethylol propane, and the like, with cyclic monomeric anhydrides such as hexahydrophthalic anhydride, methylhexahydrophthalic anhydride, and the like.

Hydroxyl Oligomers: The above acid oligomers further reacted with monofunctional epoxies such as butylene oxide, propylene oxide, and the like.

Anhydride Oligomers: The above acid oligomers further reacted with ketene.

Silane Oligomers: The above hydroxyl oligomers further reacted with isocyanato propyltrimethoxy silane.

Epoxy Oligomers: The diglycidyl ester of cyclohexane dicarboxylic acid, such as Araldite® CY-184 from Ciba Geigy, and cycloaliphatic epoxies, such as ERL®-4221, and the like from Union Carbide.

Aldimine Oligomers: The reaction product of isobutyraldehyde with diamines such as isophorone diamine, and the like.

Ketimine Oligomers: The reaction product of methyl isobutyl ketone with diamines such as isophorone diamine.

Melamine Oligomers: Commercially available melamines such as CYMEL® 1168 from Cytec Industries, and the like.

AB-Functionalized Oligomers: Acid/hydroxyl functional oligomers made by further reacting the above acid oligomers with 50%, based on equivalents, of monofunctional epoxy such as butylene oxide or blends of the hydroxyl and acid oligomers mentioned above or any other blend depicted above.

CD-Functionalized Crosslinkers: Epoxy/hydroxyl functional crosslinkers such as the polyglycidyl ether of Sorbitol DCE-358® from Dixie Chemical or blends of the hydroxyl oligomers and epoxy crosslinkers mentioned above or any other blend as depicted above.

The compositions disclosed herein may additionally contain a binder of a noncyclic oligomer, i.e., one that is linear or aromatic. Such noncyclic oligomers can include, for instance, succinic anhydride- or phthalic anhydride-derived moieties in the Acid Oligomers: such as described above.

Preferred functionalized oligomers have weight average molecular weight not exceeding about 3,000 with a polydispersity not exceeding about 1.5; more preferred oligomers have molecular weight not exceeding about 2,500 and polydispersity not exceeding about 1.4; most preferred oligomers have molecular weight not exceeding about 2,200, and polydispersity not exceeding about 1.25. Typically, compositions will comprise from about 20 to about 80 weight percent of the functionalized oligomer based on the total weight of 2,6-dioxa-7-aza-bicyclo[2.2.2]octane-containing compound in the coating. Preferably compositions will comprise from about 30 to about 70 weight percent of the functionalized oligomer based on the total weight of the 2,6-dioxa-7-aza-bicyclo[2.2.2]octane-containing compound in the coating. More preferably compositions will comprise from about 40 to about 60 weight percent of the functionalized oligomer based on the total weight of 2,6-dioxa-7-aza-bicyclo[2.2.2]octane-containing compound in the coating. Other additives also include polyaspartic esters, which are the reaction product of diamines, such as, isopherone diamine with dialkyl maleates, such as, diethyl maleate.

The coating compositions may be formulated into high solids coating systems dissolved in at least one solvent. The solvent is usually organic. Preferred solvents include aromatic hydrocarbons such as petroleum naphtha or xylenes; ketones such as methyl amyl ketone, methyl isobutyl ketone, methyl ethyl ketone or acetone; esters such as butyl acetate or hexyl acetate; and glycol ether esters such as propylene glycol monomethyl ether acetate.

The coating compositions can also contain a binder of an acrylic polymer of weight average molecular weight greater than 3,000, or a conventional polyester such as SCD®-1040 from Etna Product Inc. for improved appearance, sag resistance, flow and leveling and such. The acrylic polymer can be composed of typical monomers such as acrylates, methacrylates, styrene and the like and functional monomers such as hydroxy ethyl acrylate, glycidyl methacrylate, or gamma methacrylylpropyl trimethoxysilane and the like.

The coating compositions can also contain a binder of a dispersed acrylic component which is a polymer particle dispersed in an organic media, which particle is stabilized by what is known as steric stabilization. Hereafter, the dispersed phase or particle, sheathed by a steric barrier, will be referred to as the “macromolecular polymer” or “core”. The stabilizer forming the steric barrier, attached to this core, will be referred to as the “macromonomer chains” or “arms”.

The dispersed polymer contains about 10 to 90%, preferably 50 to 80%, by weight, based on the weight of the dispersed polymer, of a high molecular weight core having a weight average molecular weight of about 50,000 to 500,000. The preferred average particle size is 0.1 to 0.5 microns. The arms, attached to the core, make up about 10 to 90%, preferably 10 to 59%, by weight of the dispersed polymer, and have a weight average molecular weight of about 1,000 to 30,000, preferably 1,000 to 10,000. The macromolecular core of the dispersed polymer is comprised of polymerized acrylic monomer(s) optionally copolymerized with ethylenically unsaturated monomer(s). Suitable monomers include styrene, alkyl acrylate or methacrylate, ethylenically unsaturated monocarboxylic acid, and/or silane-containing monomers. Such monomers as methyl methacrylate contribute to a high Tg (glass transition temperature) dispersed polymer, whereas such “softening” monomers as butyl acrylate or 2-ethylhexylacrylate contribute to a low Tg dispersed polymer. Other optional monomers are hydroxyalkyl acrylates or methacrylates or acrylonitrile. Optionally, the macromolecular core can be crosslinked through the use of diacrylates or dimethacrylates such as allyl methacrylate or post reaction of hydroxyl moieties with polyfunctional isocyanates. The macromonomer arms attached to the core can contain polymerized monomers of alkyl methacrylate, alkyl acrylate, each having 1 to 12 carbon atoms in the alkyl group, as well as glycidyl acrylate or glycidyl methacrylate or ethylenically unsaturated monocarboxylic acid for anchoring and/or crosslinking. Typically useful hydroxy-containing monomers are hydroxy alkyl acrylates or methacrylates as described above.

The coating compositions can also contain conventional additives such as pigments, stabilizers, rheology control agents, flow agents, toughening agents and fillers. Such additional additives will, of course, depend on the intended use of the coating composition. Fillers, pigments, and other additives that would adversely effect the clarity of the cured coating will not be included if the composition is intended as a clear coating.

The coating compositions are typically applied to a substrate by conventional techniques such as spraying, electrostatic spraying, roller coating, dipping or brushing. As mentioned above atmospheric moisture may “diffuse” into the coating and cause curing, or alternatively just before the coating is applied it is mixed with an appropriate amount of water, as in a mixing spray head. Under these latter conditions it is important to apply the coating before it crosslinks. The present formulations are particularly useful as a clear coating for outdoor articles, such as automobile and other vehicle body parts. The substrate is generally prepared with a primer and or a color coat or other surface preparation prior to coating with the present compositions.

A layer of a coating composition is cured under ambient conditions in the range of 30 minutes to 24 hours, preferably in the range of 30 minutes to 3 hours to form a coating on the substrate having the desired coating properties. It is understood that the actual curing time depends upon the thickness of the applied layer and on any additional mechanical aids, such as, fans that assist in continuously flowing air over the coated substrate to accelerate the cure rate. If desired, the cure rate may be further accelerated by baking the coated substrate at temperatures generally in the range of from about 60° C. to 150° C. for a period of about 15 to 90 minutes. The foregoing baking step is particularly useful under OEM (Original Equipment Manufacture) conditions.

### EXAMPLES

In the Examples below, the following tests and procedures were used:

Film Preparation

The clearcoats were drawn down over Uniprime (ED-5000, cold-rolled steel (04X12X032)B952 P60 DIW unpolish Ecoat POWERCRON 590 from ACT Laboratories, Hillsdale, Mich.), TPO, using a 6 mil drawdown blade.

Micro-Hardness

The micro-hardness of the coatings was measured using a Fischerscope hardness tester (model HM100V, Fischer Technologies, Windsor, Conn.). The tester was set for maximum force of 100 mN ramped in series of 50, 1 second steps. The hardness was recorded in N/mm².

Swell Ratio

The swell ratio of the free films (removed from TPO) was determined by swelling in methylene chloride. The free film was placed between two layers of aluminum foil and using a LADD punch (Ladd Research, Williston, Vt.), a disc of about 3.5 mm diameter was punched out of the film. The aluminum foil was removed from either side of the free film. Using a microscope with 10× magnification and a filar lens the unswollen diameter (D_{O}) of the film measured. Four drops of methylene chloride were added to the film, the film was allowed to swell for a few seconds and then a glass slide was placed over it. The swell ratio was then calculated as:
swell ratio=(D_{S})²/(D_{O})²
Gel Fraction

Measured according to the procedure set forth in U.S. Pat. No. 6,221,494 col. 8 line 56 to col. 9 line 2 which procedure is hereby incorporated by reference.

Strike-In

Strike-in is the interaction between the basecoat and clearcoat, during wet-on-wet application. The degree of interaction is dependent upon the formulation, process parameters and/or ambient conditions. When this interaction is excessive, strike-in, or redissolving, will occur. This will result in a mottled appearance of the basecoat and a “fuzzy” appearance of the clearcoat. Thus, it is critical to minimize this strike-in to maximize appearance. The degree of strike-in can be expressed by measuring the “flop index” or “flop” of a panel. The lower the flop measurement, the greater the strike-in. The flop of samples can be measured on the same day as the basecoat-clearcoat application with a Chromavision MA100, available from DuPont, Wilmington, Del.

Unless otherwise specified, all chemicals and reagents were used as received from Aldrich Chemical Co., Milwaukee, Wis.

### Example 1

### 1-methyl-4-ethyl-7-butyl-2,6-dioxa-7-aza-bicyclo-[2.2.2]octane

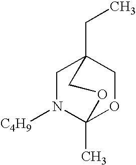

2-butyl-2-(methylaminomethyl)propanediol (96.0 g, 0,508 mol), triethyl orthoacetate (165.0 g, 1.019 mol) and toluenesulfonic acid (1.5 g) were placed in an oven dried round bottom flask quipped with a stirrer and distillation head under nitrogen. The reaction content was heated gently until ethanol distilled over. After the theoretical amount of ethanol distilled off the reaction was cooled to room temperature and then a small amount of triethylamine added to neutralize the acid. Fractional vacuum distillations (twice) afforded the material as a water clear liquid boiling point: 83.0-86.0° C. at 1.1-1.2 torr. Yield: 72.60 g (67.3%)

### Example 2

### 1-methyl-4-ethyl-7-hexyl-2,6-dioxa-7-aza-bicyclo-[2.2.2]octane

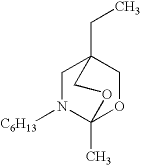

2-Hexyl-2-(methylaminomethyl)propanediol (181.675 g, 0.845 mol), triethyl orthoacetate (203.5 g, 1.25 mol) and toluenesulfonic acid (2.5 g) were placed in an oven dried round bottom flask quipped with a stirrer and distillation head under nitrogen. The reaction content was heated gently until ethanol distilled over. After the theoretical amount of ethanol distilled off the reaction was cooled to room temperature and then a small amount of triethylamine added to neutralize the acid. Fractional vacuum distillations (twice) afforded the material as a water clear liquid boiling point: 128.0-132.0° C. at 2.4 torr. Yield: 152.0 g (79.80%)

### Example 3

### 1-Buthyl-4-ethyl-7-hexyl-2,6-dioxa-7-aza-bicyclo-[2.2.2]octane

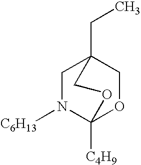

2-Hexyl-2-(methylaminomethyl)propanediol (99.13 g, 0.461 mol), ortho-n-valeric acid trimethyl ester (110.50 g, 0.682 mol) and toluenesulfonic acid (1.36 g) were placed in an oven dried round bottom flask quipped with a stirrer and distillation head under nitrogen. The reaction content was heated gently until methanol distilled over. After the theoretical amount of methanol distilled off the reaction was cooled to room temperature and then a small amount of triethylamine added to neutralize the acid. Fractional vacuum distillations (twice) afforded the material as a water clear liquid boiling point: 137.7-140.3° C. at 1.3-2.3 torr. Yield: 109.27 g (83.70%)

### Example 4

### Preparation of 1-Butyl-4-ethyl-7-octyl-2,6-dioxa-7-aza-bicyclo-[2.2.2]octane

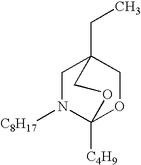

2-octyl-2-(methylaminomethyl)propanediol (88.83 g, 0.0.381 mol), ortho-n-valeric acid trimethyl ester (91.36 g, 0.564 mol) and toluenesulfonic acid (1.13 g) were placed in an oven dried round bottom flask quipped with a stirrer and distillation head under nitrogen. The reaction content was heated gently until methanol distilled over. After the theoretical amount of methanol distilled off the reaction was cooled to room temperature and then a small amount of triethylamine added to neutralize the acid. Fractional vacuum distillations (twice) afforded the material as a water clear liquid boiling.

### Example 5

### 7-butyl-4-ethyl-1-octyl-2,6-dioxa-7-aza-bicyclo-[2.2.2]octane

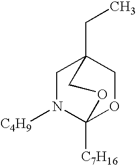

2-butyl-2-(methylaminomethyl)propanediol (26.46 g, 0.14 mol), octyl nitrile (29.20 g, 0.21 mol) and zinc acetate dihydrate (1.82 g) were placed in an oven dried round bottom flask quipped with a stirrer and a reflux condenser under nitrogen. The reaction content was heated to 140° C. for ˜44 hours.

Repeated fractional distillation afforded the desired material as a slightly impure yellow liquid, 30.88 g (60.54% yield).

### Example 6

### 1-Heptyl-4-ethyl-7-butyl-2,6-dioxa-7-aza-bicyclo-[2.2.2]octane

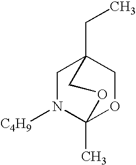

2-butyl-2-(methylaminomethyl)propanediol (37.8 g, 0.20 mol), valeronitrile (24.92 g, 0.300 mol) and cadmium acetate dihydrate (2.0 g) were placed in an oven dried round bottom flask quipped with a stirrer and a reflux condenser under nitrogen. The reaction content was heated to 130-140° C. for ˜44 hours.

Fractional distillation afforded the desired material as a light yellow liquid, 17.57 g (42.26% yield).

### Example 7

### Coating Formulation

In a glass jar 10.28 grams of 1-butyl-4-ethyl-7-hexyl-2,6-dioxa-7-aza-bicyclo[2.2.2]octane (as prepared in Example 1) was combined with 3.24 grams of propylene glycol monomethylether acetate, 1.13 grams of a 2% dibutyl tin dilaurate solution in ethyl acetate, and 0.14 grams of BYK 306 and 0.05 grams of Byk 361. To this was added 62.59 grams of a solution of 30.02 grams of Desmodur® XP 2410 (hexamethylene diisocyanate trimer available from Bayer), 28.6 grams of Desmodur® Z4470BA (isophorone diisocyanate trimer available from Bayer) and 3.97 grams n-butyl acetate. This mixture was stirred and then 0.33 grams of acetic acid was added and the mixture and stirred. The mixture was drawndown to give coatings of ˜3 mils in thickness. The coating was baked at 140° F. for 30 minutes. At seven days the coating had a Fischerscope hardness of 111 N/mm2. At 30 days the Tg (at the midpoint) was 59° C. and the gel fraction was 93%.

What is claimed is:

1. A process for producing 2,6-dioxa-7-aza-bicyclo[2.2.2]octane, said process comprising:
a) combining 2-alkyl-2-alkylaminomethyl-propane-1,3-diol with orthoester, optionally in the presence of a solvent, and optionally in the presence of an acid, to form a mixture comprising an alcohol; and
b) distilling said alcohol in an inert atmosphere to produce 2,6-dioxa-7-aza-bicyclo[2.2.2]octane.

2. The process of claim 1, further comprising after step b), neutralizing said mixture with amine and isolating said 2,6-dioxa-7-aza-bicyclo[2.2.2]octane.

3. The process of claim 1, wherein said orthoester is of the structure (RO)3-CR3 wherein R is independently C1-C20 alkyl, C6-C20 aromatic, C6-C20 alkylaromatic or C6-C20 aromaticalkyl.

4. The process of claim 3, wherein said orthoester is trimethyl orthoacetate, triethyl orthoacetate, or ortho-n-valeric acid trimethyl ester.

5. The process of claim 1, wherein said solvent is present and is toluene or xylene.

6. The process of claim 1, wherein said acid is present and is toluenesulfonic acid.

7. The process of claim 2, wherein said amine is trialkylamine of carbon chain length 1 to 20.

8. The process of claim 2, wherein said isolating is achieved by distillation, crystallization or combination thereof.

9. A process for producing 2,6-dioxa-7-aza-bicyclo[2.2.2]octane, said process comprising:
a) reacting 2-alkyl-2-alkylaminomethyl-propane-1,3-diol with nitrile in the presence of metal salt, optionally in the presence of solvent, to form a mixture comprising ammonia; and
b) removing said ammonia to produce a 2,6-dioxa-7-aza-bicyclo[2.2.2]octane.

10. The process of claim 9, further comprising isolating said 2,6-dioxa-7-aza-bicyclo[2.2.2]octane.

11. The process of claim 1 or 9, wherein the carbon chain length of said 2-alkyl-2-alkylaminomethyl-propane-1,3-diol is 1 to 20.

12. The process of claim 10, wherein said nitrile is alkyl nitrile having a carbon chain length of 1 to 20.

13. The process of claim 12, wherein said alkyl nitrile is octyl nitrile.

14. The process of claim 9, wherein said metal salt is comprised of a principal metal component, wherein said principal metal component is selected from the group consisting of scandium, titanium, vanadium, chromium, manganese, iron, cobalt, nickel, copper, zinc, and cadmium.

15. The process of claim 14, wherein said metal salt is zinc acetate dihydrate or cadmium acetate dihydrate.

16. The process of claim 1 or 9, wherein said solvent is present and is toluene or xylene.

17. The process of claim 10, wherein said isolation is achieved by distillation, crystallization or combination thereof.

18. A composition, comprising:
one or more 2,6-dioxa-7-aza-bicyclo[2.2.2]octanes of the structure: 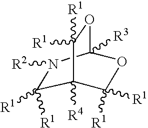
wherein each R¹, R², R³ and R⁴ can be independently C1-C20 alkyl, C6-C20 aromatic, C6-C20 alkylaromatic or C6-C20 aromaticalkyl, wherein each of R¹, R², R³ and R⁴ optionally may be substituted with hydroxyl, amino, epoxy, or carboxy groups; and a crosslinking moiety.

19. The composition of claim 18, wherein the crosslinking moiety is selected from the group consisting of isocyanate(s), epoxide(s), carboxylic acid anhydride(s), melamine(s) and silane(s).

20. A process for forming a coating composition comprising 2,6-Dioxa-7-aza-bicyclo[2.2.2]octanes of claim 18, said process comprising reacting a 2,6-Dioxa-7-aza-bicyclo[2.2.2]octanes with a crosslinking moiety.

21. The composition of claim 18, wherein the crosslinking moiety is one or more isocyanate(s), said isocyanate(s) selected from the group consisting of isocyanurate trimer of hexamethylene diisocyanate, isophorone diisocyanate, and combinations thereof.

22. A method of using the composition of claim 18 wherein said method comprises applying said composition to a substrate.

23. A coating composition made by the process of claim 20.

24. The coating composition of claim 20 wherein said composition is a component in basecoat-clearcoat system.

25. A substrate coated with the coating composition of claim 18.
